(19) United States
(12) Patent Application Publication
(10) Pub. No.: US 2014/0302065 A1
(43) Pub. Date: Oct. 9, 2014

(54) SOLUBLE UROKINASE RECEPTOR (SUPAR) IN DIABETIC KIDNEY DISEASE

(71) Applicant: THE UNIVERSITY OF MIAMI, MIAMI, FL (US)

(72) Inventors: Alessia Fornoni, Coral Gables, FL (US); Jochen Reiser, Hinsdale, IL (US); Changli Wei, Miami, FL (US); Sandra Monika Merscher-Gomez, Miami, FL (US)

(73) Assignee: THE UNIVERSITY OF MIAMI, MIAMI, FL (US)

(21) Appl. No.: 14/354,892

(22) PCT Filed: Oct. 30, 2012
(86) PCT No.: PCT/US12/62594

§ 371 Date: Apr. 28, 2014

Related U.S. Application Data
(60) Provisional application No. 61/553,414, filed on Oct. 31, 2011.

(51) Int. Cl. G01N 33/53; C07K 14/705

(52) U.S. Cl. CPC G01N 33/53; C07K 14/705
USPC 424/172.1; 436/501; 435/7.9; 435/7.1; 435/7.92

(57) ABSTRACT

Methods and compositions are provided for diagnosing and treating a diabetic kidney disease (DKD) in an individual. In aspects of the methods, the levels of soluble circulating urokinase receptor (suPAR) in the blood are measured to predict a DKD, diagnose a DKD, or provide a prognosis pertaining to a DKD. In addition, reagents, devices and kits thereof that find use in practicing the subject methods are provided.

### FIELD OF THE INVENTION

This invention pertains to the diagnosis and treatment of diabetic kidney disease.

### BACKGROUND OF THE INVENTION

Diabetic kidney disease (DKD) is the single most common cause of end stage renal disease (ESRD). The pathology and injury pattern of DKD is in many ways similar to other sclerosing diseases of the kidney such as FSGS. A persistent clinical dilemma is the lack of methods and reagents for diagnosing DKD in patients with Diabetes Mellitus. The present invention addresses these issues.

### SUMMARY OF THE INVENTION

Methods and compositions are provided for diagnosing and treating a diabetic kidney disease (DKD) in an individual. In aspects of the methods, the levels of soluble circulating urokinase receptor (suPAR) in the blood are measured to predict a DKD, diagnose a DKD, or provide a prognosis pertaining to a DKD. In addition, reagents, devices and kits thereof that find use in practicing the subject methods are provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 demonstrates that elevated circulating suPAR concentrations are associated with DKD. A: The sera from three groups of patient (N=10 each) were studied. NHS patients: patients with Normal Human Sera. DM w/o DKD patients: patients with T1DM and no microalbuminuria. DM w DKD: patients with T1D and DKD. Patients were matched for age, sex, duration of diabetes, HgA1c, and lipid profile B: Dot plot analysis of circulating suPAR concentrations demonstrating a significantly higher serum circulating suPAR level in patients with DKD (3851±746 pg/ml) when compared to DM w/o DKD (2300±576.4 pg/ml) and NHS (1689±219.6 pg/ml, p<0.001). These data demonstrate that suPAR serves as a biomarker for the diagnosis as well as for the prediction of DKD. C: circulating suPAR in subjects with glomerular disease and healthy human subjects. MN, membranous nephropathy. *P<0.05 for FSGS versus MN and preeclampsia; #P<0.001 for FSGS versus healthy, MCD relapse, and MCD remission.

FIG. 2 demonstrates that elevated circulating suPAR are observed in early DKD prior to any change in GFR. A: Historical sera from the year 2000 and 2007 was obtained from five patients with diabetes that have now progressed to diabetic nephropathy with altered renal function (31, 32, 33, 23, 28). In year 2000, two of the five patients were normolabuminuric (light gray bar), while the other three had microalbuminuria with normal renal function (dark gray). In year 2007, the two patients that were initially normoalbuminuric progressed to microalbuminuria, while those three patients that had microalbuminuria progressed to macroalbuminuria. The concentration of suPAR was higher in the sera of patients that progressed to DKD when compared to age and sex matched normal human sera (NHS) irrespectively of the time that the samples were collected, suggesting that increased circulating suPAR prior to the development of microalbuminuria may predict patients risk to progress to DKD. B: Five patients with T1D that ultimately developed progressive DKD with loss of GFR were studied. Two historical sets of sera were available for these 5 patients: one (Normo/Micro) collected when all patients had normal GFR and either microalbuminuria (n=3) or normoalbuminuria (designated on the plot as “NA”; n=2); a second one (Macro) collected 7 years later when all patients had developed macroalbuminuria. Ten normal human sera controls (NHS) matched for age and sex to the Normo/Micro group were utilized. Circulating suPAR concentrations were higher in patients with DKD already at the time they were normoalbuminuric and with normal GFR, therefore prior to the diagnosis of DKD. These data demonstrate that increased circulating suPAR in the population of patients with diabetes may represent an early biomarker of disease development and progression.

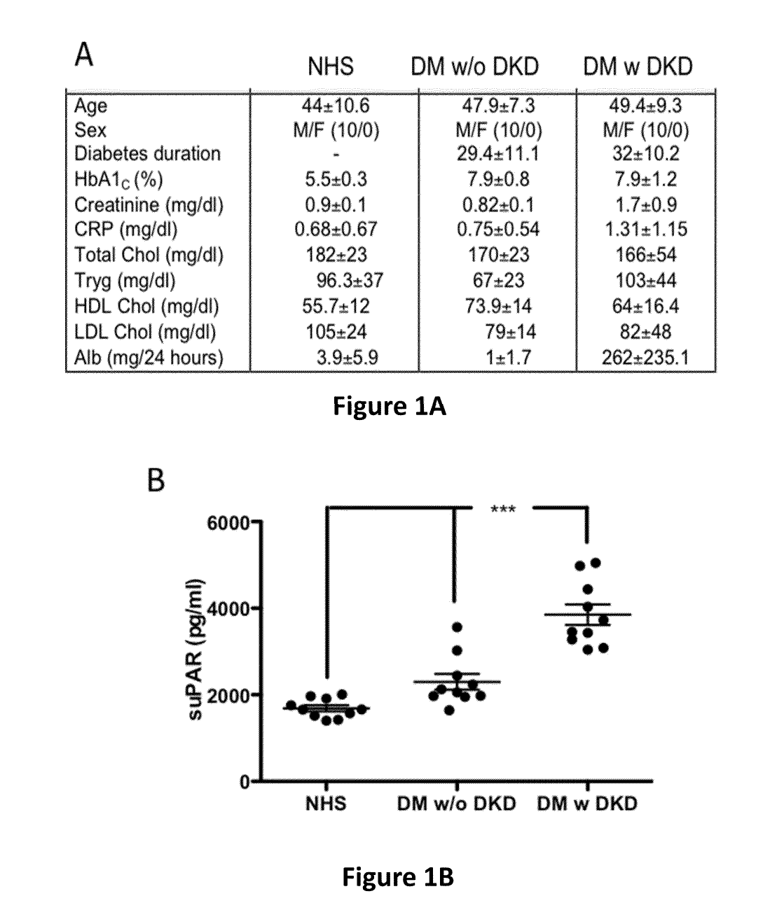

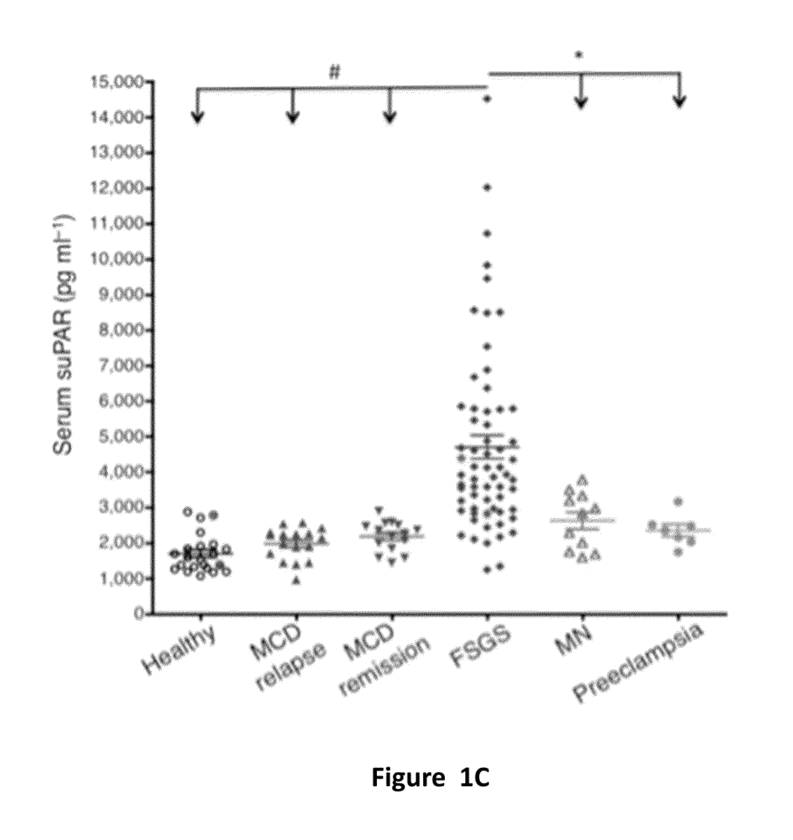

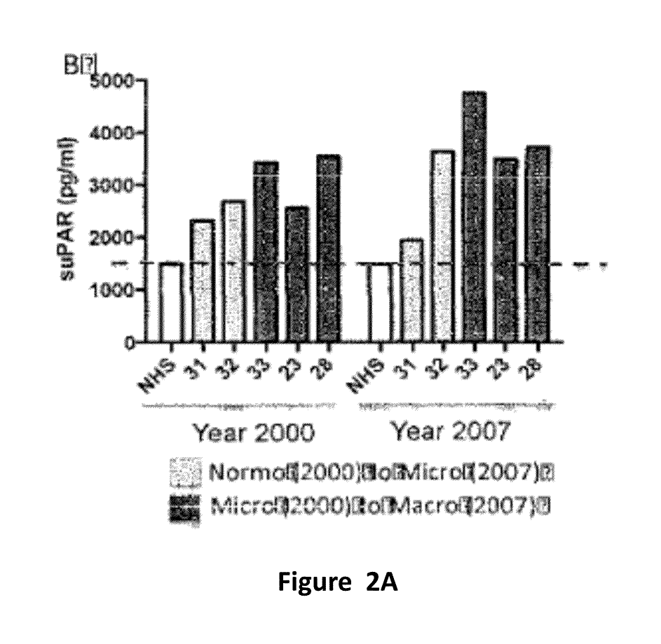

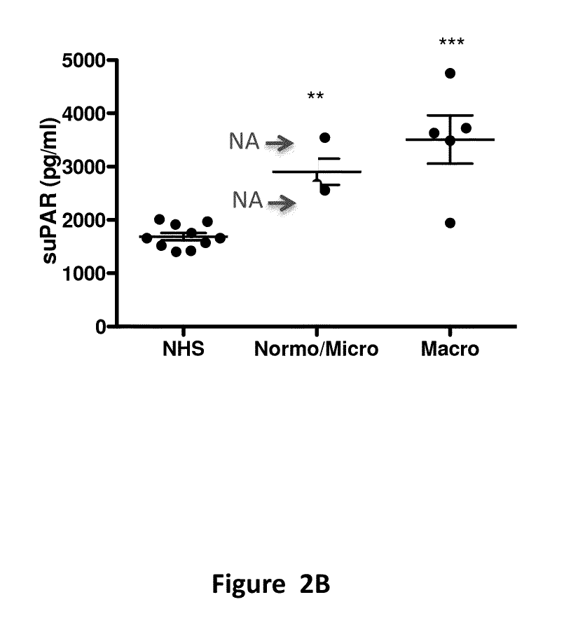

### DETAILED DESCRIPTION OF THE INVENTION

Methods and compositions are provided for diagnosing and treating a diabetic kidney disease (DKD) in an individual. In aspects of the methods, the levels of soluble circulating urokinase receptor (suPAR) in the blood are measured to predict a DKD, diagnose a DKD, or provide a prognosis pertaining to a DKD. In addition, reagents, devices and kits thereof that find use in practicing the subject methods are provided. These and other objects, advantages, and features of the invention will become apparent to those persons skilled in the art upon reading the details of the compositions and methods as more fully described below.

Before the present methods and compositions are described, it is to be understood that this invention is not limited to particular methods and compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “kidney disease” is used herein to refer to any condition that greatly reduces the function of the kidneys in removing waste products and excess fluid from the body. Symptoms of a kidney disease may include burning or difficulty during urination, an increase in the frequency of urination, passage of blood in the urine, volume retention (e.g. puffiness around the eyes, swelling of the hands and feet), pain in the small of the back just below the ribs, high blood pressure, a reduction in the glomerular filtration rate (GFR), and albuminuria or proteinuria.

By “acute kidney disease” or “acute renal failure” (ARF), it is a kidney disease in which a decline in kidney function is sudden and symptoms reveal themselves very quickly.

By “chronic kidney disease” (CKD) it is meant a kidney disease in which the decline in kidney function is slow and progressive. There are five stages of CKD. Stage 1 CKD is slightly diminished function, observed as a normal or relatively high glomerular filtration rate (GFR) (>90 mL/min/1.73 m²) and kidney damage defined as pathological abnormalities or markers of damage, including abnormalities in blood or urine test or imaging studies. Stage 2 CKD (mild CKD) presents as a mild reduction in GFR (60-89 mL/min/1.73 m²) with evidence of kidney damage defined as above. Stage 3 CKD (moderate CKD) is observed as a moderate reduction in GFR (30-59 mL/min/1.73 m²). Stage 4 CKD (severe CKD) is observed as a severe reduction in GFR (15-29 mL/min/1.73 m²). Stage 5 CKD, also known as End Stage Renal Disease (ESRD), is established kidney failure (GFR<15 mL/min/1.73 m² and requires permanent renal replacement therapy (RRT, including maintenance dialysis or renal transplantation).

By “glomerular filtration rate” or GFR it is meant the flow rate of filtered fluid through the kidney. In other words, it is the volume of fluid filtered from the renal (kidney) glomerular capillaries into the Bowman's capsule per unit time. GFR may be determined by a number of different techniques. For example, inulin or the inulin-analogon sinistrin may be injected into the plasma and its excretion in urine measured. As another example, GFR may be approximated based on determined (C_{Cr}) or estimated (eC_{Cr}) rate of creatinine clearance from the body using any convenient methodology. GFR in a normally functioning kidney is typically above 90 mL/min/1.73 m² and no proteinuria

By “proteinuria” it is meant the presence of excessive amounts of serum proteinin the urine. Proteinuria is a characteristic symptom of either renal (kidney), urinary, pancreatic distress, nephrotic syndromes (i.e., proteinuria larger than 3.5 grams per day), eclampsia, toxic lesions of kidneys, and it is frequently a symptom of diabetes mellitus. With severe proteinuria general hypoproteinemia can develop and it results in diminished oncotic pressure (ascites, edema, hydrothorax). Nonlimiting examples of methods for detecting proteinuria include a urinalysis for protein, e.g. a quantitative protein determination in a timed urine collection or the ratio of protein levels relative to creatinine levels in a random urine collection, or by a foamy appearance or excessive frothing of the urine.

By “albuminuria” it is meant a type of proteinuria in which the protein albumin is detectable in urine. Tests for albuminuria are typically more sensitive than tests for proteinuria. As such, in some instances, an individual may test positive for albuminuria but negative for proteinuria. Nonlimiting examples of methods for measuring albuminuria include a quantitative albumin determination in a timed urine collection or the ratio of albumin levels relative to creatinine levels in a random urine collection (the albumin/creatinine ratio (ACR)).

By “normoalbuminuria” it is meant having a substantially normal level of albumin in the urine. The presence and level of albumin protein in urine may be determined by a urine test, in which the concentration of albumin is measured in a 24-hour urine collection, or a spot test. Normoalbuminuria is characterized by a level of albumin of about 30 mg or less in a 24 hour collection (30 mg or less/day). In some instances, normoalbuminuria is defined based on the albumin/creatinine ratio (ACR), which is the amount of albumin in the sample compared to the concentration of creatinine in the sample. In such instances, normoalbuminuria is defined as an ACR of about 30 μg or less albumin/mg creatinine (“30 μg or less/mg”).

By “microalbuminuria” it is meant a condition caused by increased permeability for albumin in the renal glomerulus. Microalbuminuria is defined as a level of albumin of 30 to 300 mg in a 24 hour urine collection (30-300 mg/24 hours); or as an ACR of 30 to 300 μg albumin/mg creatinine (“30-300 μg/mg”).

By “macroalbuminuria” it is meant a condition caused by an abnormally high permeability for albumin in the renal glomerulus. Macroalbuminuria is characterized as a level of albumin of 300 mg or more in a 24 hour urine collection (more than 300 mg/24 hours); or as an ACR of 300 μg albumin nor more per mg creatinine (“300 μg or more/mg”).

By “diabetes” it is meant a metabolic disease that occurs when the pancreas does not produce enough of the hormone insulin to regulate blood sugar (“type 1 diabetes mellitus”) or, alternatively, when the body cannot effectively use the insulin it produces (“type 2 diabetes mellitus”). Type 1 diabetes, also known as insulin dependent diabetes mellitus (IDDM), results from the destruction or dysfunction of β cells by the cells of the immune system. Symptoms include polyuria (frequent urination), polydipsia (increased thirst), polyphagia (increased hunger), and weight loss. T1D is fatal unless treated with insulin and must be continued indefinitely, although many people who develop the disease are otherwise healthy and treatment need not significantly impair normal activities. Exercising regularly, eating healthy foods and monitoring blood sugar may also be recommended. Other medications may be prescribed as well, including one or more of the following: medications to slow the movement of food through the stomach (e.g. pramlintide), high blood pressure medications, cholesterol-lowering drugs. Type 2 diabetes, also known as non-insulin dependent diabetes mellitus (NIDDM), is associated with resistance to insulin in peripheral tissues (such as skeletal muscles and liver) and by a gradual decline in 13 cell function and numbers over time, as the 13 cells develop resistance to insulin as well. As a result, in T2D the pancreas does not make enough insulin to keep blood glucose levels normal. Symptoms include hyperglycemia (high blood sugar), diabetic ketoacidosis (increased ketones in urine), and hyperosmolar hyperglycemic nonketotic syndrome. Therapy may include blood sugar monitoring; healthy eating; regular exercise; diabetes medication that lowers glucose production (e.g. metformin, sitagliptin, saxagliptin, repaglinide, nateglinide, exenatide, liraglutide), that stimulates the pancreas to produce and release more insulin (e.g. glipizide, glyburide, glimepiride), and/or that blocks the action of enzymes that break down carbohydrates or make tissues more sensitive to insulin (e.g. pioglitazone); and insulin therapy.

“Diabetic kidney disease” and “Diabetic nephropathy” are used interchangeably herein to mean a chronic kidney disease caused by or associated with diabetes. Symptoms of diabetic kidney disease include the occurrence of microalbuminuria or macroalbuminuria, or the progressive decline of GFR in a normoalbuminuric individual with any form of diabetes.

By “early stage” diabetic kidney disease, or “early stage” diabetic nephropathy it is meant diabetic kidney disease with normoalbuminuria or microalbuminuria and normal or high GFR, i.e. a GFR of 90 mL/min/1.73 m² or more.

By “progressive” diabetic kidney disease or “progressive” diabetic nephropathy it is meant diabetic kidney disease with macroalbuminuria or with stage 2 chronic kidney disease (CKD) or worse, i.e., a glomerular filtration rate (GFR) of less than 90 cc/min/1.73 m².

“Diagnosis” as used herein generally includes a prediction of a subject's susceptibility to a disease or disorder, determination as to whether a subject is presently affected by a disease or disorder, prognosis of a subject affected by a disease or disorder (e.g., predicting whether the disease or disorder will progress to a more sever stage), classification of the subject's disease or disorder into a subtype of the disease or disorder, prediction of a subject's responsiveness to treatment for the disease or disorder (e.g., positive response, a negative response, no response at all) and use of therametrics (e.g., monitoring a subject's condition to provide information as to the effect or efficacy of therapy).

“Prognosis” as used herein generally includes a prediction of the course of disease progression and/or disease outcome, and may include the expected duration, the function, and a description of the course of the disease.

By “disease severity” is meant relative stage of disease progression. Disease severity may be correlated with the impact the disease may have on the patient's overall health or the risk of patient death as a result of disease. The severity of the disease may affect decisions relating to patient treatment subsequent to diagnosis.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

By “comprising” it is meant that the recited elements are required in the composition/method/kit, but other elements may be included to form the composition/method/kit etc. within the scope of the claim. For example, a suPAR polypeptide that comprises uPAR amino acid sequence corresponding to, e.g. residues 88-277 of human uPAR or, e.g. residues 1-88 of human uPAR, may comprise uPAR amino acid sequence in addition to that sequence.

By “consisting essentially of”, it is meant a limitation of the scope of composition or method described to the specified materials or steps that do not materially affect the basic and novel characteristic(s) of the subject invention. For example, a suPAR polypeptide “consisting essentially of” a disclosed sequence has the amino acid sequence of the disclosed sequence plus or minus about 10 amino acid residues at the boundaries of the sequence based upon the full length parent human uPAR sequence from which it was derived, e.g. about 10 residues, 9 residues, 8 residues, 7 residues. 6 residues, 5 residues, 4 residues, 3 residues, 2 residues or about 1 residue less than the recited bounding amino acid residue, or about 1 residue, 2 residues, 3 residues, 4 residues, 5 residues, 6 residues, 7 residues, 8 residues, 9 residues, or 10 residues more than the recited bounding amino acid residue.

By “consisting of”, it is meant the exclusion from the composition, method, or kit of any element, step, or ingredient not specified in the claim. For example, a suPAR polypeptide “consisting of” a disclosed sequence consists only of the disclosed amino acid sequence.

### Methods and Compositions

As summarized above, aspects of the subject invention provide methods of diagnosing a diabetic kidney disease in an individual, i.e. a human individual, with diabetes. By a “diabetic kidney disease” it is meant a chronic condition caused by or associated with diabetes in which the function of the kidneys in removing waste products and excess fluid from the body declines slowly and progressively. By “diagnosing a diabetic kidney disease” it is meant determining whether a human subject with diabetes has a kidney disease or will develop a kidney disease (i.e. prognosing a diabetic kidney disease), including determining the severity of the disease, predicting the progression of the disease, monitoring the progression of the disease or responsiveness to a course of treatment therefore, etc. In one aspect, the subject invention provides methods for determining whether an individual is suffering from a diabetic kidney disease and/or determining the severity of the diabetic kidney disease, e.g. determining whether the individual has early stage diabetic kidney disease or progressive diabetic kidney disease. In another aspect, the subject invention provides methods for prognosing a diabetic kidney disease, e.g. predicting the onset of early stage diabetic kidney disease, predicting the progression of the diabetic kidney disease, predicting the onset of progressive diabetic kidney disease. In yet another aspect, the subject invention provides methods for monitoring a diabetic kidney disease, e.g. monitoring progression of the diabetic kidney disease, determining responsiveness of the diabetic kidney disease to treatment, etc. In yet another aspect, the subject invention provides methods for treating a diabetic kidney disease, e.g. by diagnosing the diabetic kidney disease, and determining a treatment based on the diagnosis.

In determining whether an individual with diabetes has a diabetic kidney disease, determining the severity of the diabetic kidney disease, prognosing a diabetic kidney disease, monitoring a diabetic kidney disease, treating a diabetic kidney disease, etc., a sample of body fluid from the individual with diabetes is assayed to detect the amount of soluble urokinase-type plasminogen activator receptor (suPAR) analyte in the sample. By “soluble urokinase-type plasminogen activator receptor” or “suPAR” it is meant a polypeptide that is the soluble form of the membrane-bound receptor for urokinase (uPA), urokinase-type plasminogen activator receptor (uPAR).

uPAR, also known as “CD87”, is a glycosylphosphatidylinositol (GPI)-anchored cell-surface protein encoded by the PLAUR gene. The amino acid sequence for uPAR precursor protein may be found at Genbank Accession No. NM_{—}002659.3 (SEQ ID NO:1), and for the mature polypeptide at Genbank Accession No. NP_{—}002650.1 (SEQ ID NO:2). uPAR is made up of three domains denoted uPAR(I), uPAR(II), and uPAR(III), uPAR(I) being connected to uPAR(II) by a first linker region, uPAR(II) being connected to uPAR(III) by a second linker region, and uPAR(III) being anchored to the cell membrane by a juxtamembrane GPI domain (Ploug et al, 1991; crystal structure disclosed in Llinas et al, 2005). uPAR(I-III) can be cleaved by uPA in the first linker region (i.e. between domains I and II), liberating uPAR(I) (consisting essentially of about amino acids 1-89 of SEQ ID NO:2) and leaving the cleaved form uPAR(II-III) on the cell surface (Hoyer-Hansen et al, 1992; Zhou et al, 2000). Additionally, uPAR can be cleaved by proteases at its GPI anchor, shedding suPAR(I-III) (consisting essentially of about amino acids 1-281 of SEQ ID NO:2) or—if already cleaved at linker 1 by uPA—suPAR(II-III) (consisting essentially of about amino acids 90-281 of SEQ ID NO:2) from the cell surface. Soluble forms of uPAR, i.e. suPAR(I-III) (molecular weight of approximately 30.7 kDa), suPAR(II-III) (molecular weight of approximately 21 kDa) and uPAR(I) (molecular weight of approximately 10.4 kDa) have been detected in various body fluids (Piironen et al. Specific immunoassays for detection of intact and cleaved forms of the urokinase receptor. Clin Chem 2004; 50:2059-68; Hoyer-Hansen and Lund. Urokinase receptor variants in tissue and body fluids. Adv. Clin. Chem 2007; 44:65-102).

By “soluble urokinase-type plasminogen activator receptor analyte” or “suPAR analyte” it is meant suPAR polypeptides, e.g. suPAR(I-III), suPAR(II-III), and uPAR(I) and suPAR fragments thereof, including suPAR polypeptide fragments and suPAR peptide fragments. As used herein, the term “fragment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. As used herein, the term “peptide fragments” is restricted to mean an amino acid fragment of about 40 amino acids in length or less.

As demonstrated in the Examples below, the inventors have discovered that elevated levels of suPAR analyte in body fluid correlates with the presence of diabetic kidney disease and an increased risk of developing a diabetic kidney disease. For example, as demonstrated in the Examples below, an increase of about 1.5-fold or more, e.g. 2-fold or more, 2.5-fold or more, 3-fold or more, 4-fold or more, or 5-fold or more in suPAR levels over suPAR levels observed in healthy individuals is indicative of diabetic kidney disease or an increased risk in developing a diabetic kidney disease. Put another way, suPAR analyte level of about 3000 pg/ml or more, e.g. 3500 pg/ml or more, 4000 pg/ml or more, 4500 pg/ml or more, or 5000 pg/ml or more, in a body fluid sample from a diabetic individual is diagnostic of diabetic kidney disease; and that a suPAR analyte level of about 3000 pg/ml to about 4000 pg/ml in a body fluid sample of a diabetic individual is diagnostic of diabetic kidney disease, while a suPAR analyte level of about 4000 pg/ml or more in a body fluid sample if a diabetic individual is diagnostic of progressive diabetic kidney disease. As another example, the inventors have discovered that about 3000 pg or more suPAR analyte per ml of body fluid sample, e.g. 3500 pg/ml or more, 4000 pg/ml or more, 4500 pg/ml or more, or 5000 pg/ml or more, is prognostic of the development of diabetic kidney disease in an individual has normal glomerular filtration rate (GFR) and normoalbuminuria, and that 4000 pg or more suPAR analyte per ml of body fluid sample is prognostic of the development of progressive diabetic kidney disease in an individual with normoalbuminuria or microalbuminuria. In other words, suPAR analyte is a biomarker for diabetic kidney disease. The terms “marker” or “biomarker” are used interchangeably herein and refer to DNA, RNA (including mRNA, rRNA, tRNA and tmRNA), nucleotides, nucleosides, analogs, polynucleotides, peptides and any combinations thereof.

In practicing the subject methods, suPAR analyte is detected in a sample of body fluid. By “body fluid” or “bodily fluid” is meant a naturally occurring fluid of the human body such as blood (e.g. serum, plasma), urine, mucus, sputum, saliva, cerebrospinal fluid, or lymph, particularly blood or blood products and urine. Typically, a sample volume of between about 1 μl to about 2,000 μl is sufficient for determining the level of a suPAR analyte. Generally, the sample volume will range from about 10 μl to about 1,750 μl, from about 20 μl to about 1,500 μl, from about 40 μl to about 1,250 μl, from about 60 μl to about 1,000 μl, from about 100 μl to about 900 μl, from about 200 μl to about 800 μl, from about 400 μl to about 600 μl. Any convenient method for producing a fluid sample may be employed. A sample source of particular interest is a blood sample. By a “blood sample” it is meant a volume of whole blood or fraction thereof, e.g., serum, plasma, etc. In some such embodiments, the method employs drawing venous blood by skin puncture (e.g., finger stick, venipuncture). In some embodiments, the cells in the sample are lysed or otherwise removed from the sample to prepare a plasma sample. In some embodiments, both cells and clotting factors, e.g. fibrinogen, clotting proteins, etc., are removed from the sample to prepared a serum sample, e.g. by allowing the blood to clot, e.g. in a clotting or serum separator tube, and centrifuging the serum away from the clotted blood.

The fluid that is collected is either assayed at the time of collection or stored, e.g. at 4° C., at −20° C., at −60° C., at −80° C., until assayed. The fluid may be assayed in crude form. Alternatively, the fluid may be fractionated to purify the suPAR analyte based upon size, charge, mass, or other physical characteristic prior to measuring protein concentration. If fractionation is employed, the fractionation technique may or may not employ native or non-denaturing conditions. Whether fractionation is carried out under denaturing or non-denaturing conditions depends on the particular manner in which the subject polypeptide is detected, e.g. whether or not a non-denatured form is required for detection, where representative detection methods are described in greater detail below. Typically, the non-denaturing conditions are ‘native’ conditions. By ‘native’ conditions is meant fractionation by a process that substantially preserves the conformation and folding of the low molecular fragment species in the sample. Native conditions are those conditions that do not denature proteins. A variety of non-denaturing fractionation means are known to those of skill in the art, where one means of interest is gel filtration high performance liquid chromatography (HPLC). Alternatively, fractionation may be carried out under non-native, e.g. denaturing conditions, such as sodium dodecylsulfate-polyacrylamide gel electrophoresis (SDS-PAGE). One or more fractions are then assayed to quantitate the amount of protein.

Once the patient derived sample is obtained, the sample is assayed to determine the level of suPAR analyte. In some instances, the subject sample may be treated in a variety of ways so as to enhance detection of suPAR analyte. For example, where the sample is blood, the red blood cells may be removed from the sample (e.g., by centrifugation) prior to assaying. Such a treatment may serve to reduce the non-specific background of detecting the level of a suPAR using an affinity reagent. Detection of suPAR analyte may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification). In some embodiments, the pH of the test and control samples will be adjusted to, and maintained at, a pH which approximates neutrality (i.e. pH 6.5-8.0). Such a pH adjustment will prevent suPAR analyte complex formation, thereby providing a more accurate quantitation of the level of suPAR analyte in the sample.

Any convenient method for assessing a fluid sample for polypeptides or fragments thereof may be employed to detect suPAR analyte in the subject methods. The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. In some instances, full-length suPAR polypeptide may be detected. In some instances, suPAR polypeptide fragments or suPAR peptides, i.e. suPAR fragments about 40 amino acids in length or less, may be detected.

For example, the sample may be assessed for suPAR analyte using a suPAR analyte-specific affinity reagent, also referred to interchangeably herein as a “suPAR analyte affinity reagent”. By “affinity reagent” it is meant a reagent having an analyte binding domain, moiety or component that has a high binding affinity and binding specificity for the analyte, in this instance, suPAR analyte, i.e. a suPAR polypeptide or a fragment or peptide thereof. By “high binding affinity” is meant a binding affinity of at least about 10⁻⁴ M, usually at least about 10⁻⁶ M or higher, e.g., 10⁻⁹M or higher. The affinity reagent may be any of a variety of different types of molecules, so long as it exhibits the requisite binding affinity for the target protein when present as tagged affinity ligand. In some instances, the affinity reagent has a high binding affinity for the suPAR analyte, i.e., an epitope within the suPAR polypeptide or fragment thereof. In some instances, the affinity reagent has a high binding affinity for a suPAR peptide, i.e. a peptide of the suPAR polypeptide. By “high binding specificity” and “binds specifically” is meant high avidity and/or high affinity binding of an affinity reagent to a specific antigen. For example, antibody binding to its epitope on this specific antigen is stronger than binding of the same antibody to any other epitope, particularly those which may be present in molecules in association with, or in the same sample, as the specific antigen of interest. Affinity reagents which bind specifically to a suPAR polypeptide of interest may be capable of binding other polypeptides at a weak, yet detectable, level (e.g., 10% or less of the binding shown to the polypeptide of interest). Such weak binding, or background binding, is readily discernible from the specific affinity reagent binding to the polypeptide of interest, e.g., by use of appropriate controls. In the present instance, because suPAR is a cleavage product of the uPAR protein, the suPAR analyte-specific affinity reagent may have high specificity for an epitope found in both suPAR and uPAR. In other words, in some instance, the suPAR analyte-specific affinity reagent will be specific for both suPAR and uPAR. In other instances, the suPAR analyte-specific affinity reagent will have specificity for only suPAR, e.g. it will be specific for an epitope found in suPAR and not uPAR, e.g. an epitope created by the cleavage of uPAR.

An affinity reagent may be a small molecule ligand or large molecule ligand. By small molecule ligand is meant a ligand ranging in size from about 50 to about 10,000 daltons, usually from about 50 to about 5,000 daltons and more usually from about 100 to about 1000 daltons. By large molecule is meant a ligand ranging in size from about 10,000 daltons or greater in molecular weight.

The small molecule may be any molecule, as well as binding portion or fragment thereof, that is capable of binding with the requisite affinity and specificity to the target protein. Generally, the small molecule is a small organic molecule that is capable of binding to the target analyte of interest. The small molecule will include one or more functional groups necessary for structural interaction with the target analyte, e.g., groups necessary for hydrophobic, hydrophilic, electrostatic or even covalent interactions. Where the target analyte is a protein, e.g. a suPAR polypeptide, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. The small molecule may also comprise a region that may be modified and/or participate in covalent linkage to a label component, a substrate surface, or other entity, depending on the particular assay protocol being employed, without substantially adversely affecting the small molecule's ability to bind to its target analyte.

Small molecule affinity ligands often comprise cyclical carbon or heterocyclic structures and/or aromatic or polyaromatic structures substituted with one or more of the above functional groups. Also of interest as small molecules are structures found among biomolecules, including peptides, saccharides, fatty acids, steroids, purines, pyrimidines, derivatives, structural analogs or combinations thereof. Such compounds may be screened to identify those of interest, where a variety of different screening protocols are known in the art.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e. a compound diversity combinatorial library. When obtained from such libraries, the small molecule employed will have demonstrated some desirable affinity for the protein target in a convenient binding affinity assay. Combinatorial libraries, as well as methods for the production and screening, are known in the art and described in: 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Alternatively, the affinity reagent can also be a large molecule. Of particular interest as large molecule affinity ligands are antibodies, as well as binding fragments and mimetics thereof, with affinity and specificity for an antigenic fragment of suPAR. By “antigenic fragment” of suPAR is meant a portion of suPAR which is capable of binding an antibody generated by immunization of a mammal with suPAR or a fragment thereof. Preferably, the antibodies which specifically bind an epitope of the isolated antigenic fragment will also bind the same epitope in the context of the native protein from which the fragment was derived. Examples of antibodies with specificity for suPAR analyte known in the art include antibodies ATN615 (Li et al. An anti-urokinase plasminogen activator receptor (uPAR) antibody: crystal structure and binding epitope. J Mol Biol. 2007 Jan. 26; 365(4):1117-29); IIIF10 and HD13.1 (Kotzsch et al. New ELISA for quantitation of human urokinase receptor (CD87) in cancer. Int J Oncol. 2000 October; 17(4):827-34); R2, R3, R5, R9, and R23 (Piironen et al. Specific immunoassays for detection of intact and cleaved forms of the urokinase receptor. Clin Chem 2004; 50:2059-68); and the antibodies disclosed in Haastrup et al. (Soluble urokinase plasminogen activator receptor during allogeneic stem cell transplantation. Sc and J Immunol. 2011 April; 73(4):325-9), Lönnkvist et al. (Blood chemistry markers for evaluation of inflammatory activity in Crohn's disease during infliximab therapy. Scand J Gastroenterol. 2011 April; 46(4):420-7), Gao et al. (Detection of soluble urokinase receptor by immunoradiometric assay and its application in tumor patients. Thromb Res. 2001 Apr. 1; 102(1):25-31) and PCT Publication No. WO 2010/054189, the full disclosures of which are incorporated herein by reference.

Where antibodies are the affinity ligand, they may be a polyclonal composition, i.e. a heterogeneous population of antibodies differing by specificity. Alternatively, they may be monoclonal compositions, i.e. a homogeneous population of identical antibodies that have the same specificity for the target protein. As such, the affinity ligand may be either a monoclonal and polyclonal antibody. In yet other embodiments, the affinity ligand is an antibody binding fragment or mimetic, where these fragments and mimetics have the requisite binding affinity for the target protein. For example, antibody fragments, such as Fv, F(ab)₂, Fab′ and Fab may be prepared by cleavage of the intact protein, e.g. by protease or chemical cleavage. Also of interest are recombinantly produced antibody fragments, such as single chain antibodies or scFvs, where such recombinantly produced antibody fragments retain the binding characteristics of the above antibodies. Such recombinantly produced antibody fragments generally include at least the VH and VL domains of the subject antibodies, so as to retain the binding characteristics of the subject antibodies. These recombinantly produced antibody fragments or mimetics of the subject invention may be readily prepared using any convenient methodology, such as the methodology disclosed in U.S. Pat. Nos. 5,851,829 and 5,965,371; the disclosures of which are herein incorporated by reference.

The above described antibodies, fragments and mimetics thereof may be obtained from commercial sources and/or prepared using any convenient technology, where methods of producing polyclonal antibodies, monoclonal antibodies, fragments and mimetics thereof, including recombinant derivatives thereof, are known to those of the skill in the art.

Also suitable for use as binding domains are polynucleic acid aptamers. Polynucleic acid aptamers may be RNA oligonucleotides which may act to selectively bind proteins, much in the same manner as a receptor or antibody (Conrad et al., Methods Enzymol. (1996), 267 (Combinatorial Chemistry), 336-367).

In some instances, the affinity reagent may be detectably labeled, e.g. to facilitate detection. By “detectably labeled affinity reagent” and “detectably labeled antibody” it is meant an affinity reagent, e.g., antibody (or antibody fragment which retains binding specificity), having an attached detectable label. The detectable label may be attached by chemical conjugation, but where the label is a polypeptide, it could alternatively be attached by genetic engineering techniques. Methods for production of detectably labeled proteins are well known in the art. Detectable labels may be selected from a variety of such labels known in the art, but normally are radioisotopes, fluorophores, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin), methods for labeling antibodies, and methods for using labeled antibodies to detect an antigen (such as suPAR or suPAR fragments) are well known in the art (see, for example, Harlow and Lane, eds. (Antibodies: A Laboratory Manual (1988) Cold Spring Harbor Laboratory Press, Cold Spring Harbor, N.Y.)).

Any convenient assay protocol may be employed. For example, the assay may be performed in solution. As another example, the assay may be performed on a solid (insoluble) support (e.g. polystyrene, nitrocellulose, beads, etc.). Examples of assay formats include ELISAs (enzyme-linked immunosorbent assays; see, for example, the SUPARNOSTIC® ELISA kit (ViroGates), Kotzsch et al. Int J Oncol. 2000 October; 17(4):827-34, and Ronne et al. J Immunol Methods. 1994 Jan. 3; 167(1-2):91-101 for ELISAs for the detection of suPAR, and Piironen et al. Clin Chem 2004; 50:2059-68 and Henic et al. Clin Cancer Res. 2008 Sep. 15; 14(18):5785-93 for time-resolved fluorescence assays for the detection of suPAR(I-III), suPAR(II-III), suPAR(I-III)+suPAR(II-III), and uPAR(I)); IRMAs (immunoradiometric assays; see, for example, Gao et al. Thromb Res. 2001 Apr. 1; 102(1):25-31 for immunoradiometric assays to detect suPAR); and RIAs (radioimmunoassays), using any standard methods (e.g., as described in Current Protocols in Immunology, Coligan et al., ed.; John Wiley & Sons, New York, 1992). Typically, the assay will be performed in the presence of a control, e.g. a positive control or a negative control. For example, in certain embodiments, a series of standards, containing known concentrations of suPAR may be assayed in parallel with the samples or aliquots thereof to serve as positive controls. Furthermore, in certain embodiments, each sample and standard will be added to multiple wells so that mean values can be obtained for each.

For example, where the assay is performed in solution, the test and control samples may each incubated with a suPAR analyte affinity reagent for a time period sufficient to allow formation of analyte and affinity reagent complexes in solution, preferably between about 1 minute up to 24 hrs, or more. As previously noted, the affinity reagent may include a detectable label (e.g. radionuclide, fluorescer, or enzyme). The sample may then be treated to separate the analyte and affinity reagent complexes from excess, unreacted affinity reagent, for example by addition of anti-affinity reagent (e.g., anti-immunoglobulin antiserum) followed by centrifugation (e.g., 1000×g for 7 min) to precipitate the analyte and affinity reagent complexes, or by binding to an affinity surface such as a second, unlabelled suPAR analyte affinity reagent (e.g., antibody) fixed to a solid substrate such as Sepharose or a plastic well. Detection of affinity reagent bound to a suPAR analyte may be achieved in a variety of ways well known in the art. If necessary, a substrate for the detectable label may be added to the sample.

As another example, where the assay uses a solid support, the support may have an affinity reagent capable of specifically binding suPAR analyte, where the affinity reagent is bound to the support surface. The affinity reagent facilitates the stable, wash-resistant binding of a suPAR analyte present in the sample to the solid support. The insoluble supports may be any compositions to which affinity reagents, such as antibodies or fragments and mimetics thereof can be bound, which is readily separated from soluble material, and which is otherwise compatible with the overall method of measuring a suPAR analyte in the sample. The surface of such supports may be solid or porous and of any convenient shape. Examples of suitable insoluble supports to which the affinity reagent is bound include beads, membranes, and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples. Suitable affinity reagents include antibodies, or fragments and mimetics thereof, which specifically bind a suPAR analyte, or anti-idiotype antibodies, or fragments and mimetics thereof, which specifically bind to the anti-suPAR analyte-antibody. Methods for binding affinity reagents (e.g., antibodies, or fragments and mimetics thereof) to solid supports are well known in the art. After binding of the affinity reagent to the support, the support may be treated with a blocking agent, which binds to the support in areas not occupied by the affinity reagent. Suitable blocking agents include non-interfering proteins such as bovine serum albumin, casein, gelatin, and the like. Alternatively, several detergents at non-interfering concentrations, such as Tween, NP40, TX100, and the like may be used. Such blocking treatment reduces nonspecific binding. Alternatively, the solid support itself may bind a suPAR analyte directly through the charged properties of the support surface, thus taking advantage of the charged nature of a suPAR analyte molecule.

The test and control samples (if used) are each incubated with the solid support for a time sufficient for binding of suPAR analyte to the affinity reagent. Generally from about 0.001 to 1 ml of sample, diluted or otherwise, is sufficient, usually about 0.01 ml sufficing. The incubation time should be sufficient for suPAR analyte to bind the insoluble first affinity reagent. Generally, from about 0.1 to 3 hr is sufficient, usually 1 hr sufficing. After incubation, the reacted samples may be washed to remove unbound or non-specifically bound material. Generally, a dilute non-ionic detergent medium at an appropriate pH, generally 7-8, may be used as a wash medium. An isotonic buffer, such as phosphate-buffered saline, may be employed in the washing step. From one to six washes may be employed, with sufficient volume to thoroughly wash non-specifically bound proteins present in the sample. Preferably, the washing step will not cause dissociation of suPAR analyte and affinity reagent complexes.

A second affinity reagent which specifically binds suPAR analyte, e.g. an anti-suPAR analyte antibody, or fragment or mimetic thereof which preferably binds to a suPAR epitope different from the epitope bound by the first affinity reagent, is then incubated with the suPAR analyte-affinity reagent complexes. The concentration of the second affinity reagent will generally be about 0.1 to 50 ug/ml, preferably about 1 ug/ml. The solution containing the second antibody is generally buffered in the range of about pH 6.5-9.5. The incubation time should be sufficient for the second affinity reagent to bind available molecules. Generally, from about 0.1 to 3 hr is sufficient, usually 1 hr sufficing. After the second affinity reagent has bound, the insoluble support is generally again washed free of non-specifically bound second receptor, essentially as described for prior washes. After non-specifically bound material has been cleared, the signal produced by the bound conjugate is detected by conventional means.

The bound conjugate may be detected by any convenient method. For example, the second affinity reagent used to detect suPAR analyte bound to the support may be detectably labeled to facilitate direct or indirect detection of suPAR analyte-first affinity reagent-second affinity reagent complexes. Examples of labels which permit direct measurement of immunocomplexes include radiolabels, such as 3H or 125I, fluorescers, dyes, beads, chemilumninescers, colloidal particles, and the like. Examples of labels which permit indirect measurement of binding include enzymes where the substrate may provide for a colored or fluorescent product. In some embodiment, the second affinity reagent (e.g., antibody or fragment and mimetic thereof) is labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such affinity reagent-enzyme conjugates are readily produced by techniques known to those skilled in the art.

Alternatively, a third detectably labeled affinity reagent (e.g., antibody, or fragment and mimetic thereof) which specifically binds the second affinity reagent may be used to detect the suPAR analyte-first affinity reagent-second affinity reagent complexes. Examples of third affinity reagent/second affinity reagent-specific molecule pairs include antibody/anti-antibody and avidin (or streptavidin)/biotin. Since the resultant signal is thus amplified, this technique may be advantageous where only a small amount of a suPAR analyte is present in the sample. An example is the use of a labeled antibody specific to the second antibody. The volume, composition and concentration of the third affinity reagent solution provides for measurable binding to the suPAR analyte already bound to the second affinity reagent. Generally, the same volume as that of the sample is used: from about 0.001 to 1 ml is sufficient, usually about 0.1 ml sufficing. The concentration will generally be sufficient to saturate the suPAR analyte potentially bound to second reagent.

Where an enzyme conjugate is used for detection, an appropriate enzyme substrate is provided so a detectable product is formed. More specifically, where a peroxidase is the selected enzyme conjugate, a preferred substrate combination is H2O2 and O-phenylenediamine which yields a colored product under appropriate reaction conditions. Appropriate substrates for other enzyme conjugates such as those disclosed above are known to those skilled in the art. Suitable reaction conditions as well as means for detecting the various useful conjugates or their products are also known to those skilled in the art. For the product of the substrate O-phenylenediamine for example, light absorbance at 490-495 nm is conveniently measured with a spectrophotometer.

As another example of an assay format, suPAR analyte may be detected by using a competitive binding assay. The test and control samples are incubated with the anti-suPAR analyte affinity reagent, e.g. as described above, to allow for formation of suPAR analyte-affinity reagent complexes. The affinity reagent may be fixed to a solid surface or in solution. After washing to remove unbound material from the precipitated suPAR analyte-affinity reagent complexes or from the solid support (if any) to which the antibody is fixed, the samples are then incubated with a standard amount of competitive suPAR, competitive recombinant hybrid suPAR, or competitive suPAR fragment which retains the ability to compete with a native suPAR analyte for binding to the anti-suPAR analyte affinity reagent. In some instances, the competitive suPAR reagent may be detectably labeled to facilitate detection. In other words, detectably labeled suPAR, detectably labeled recombinant hybrid suPAR, or a detectably labeled fragment of suPAR may be used. By “detectably labeled suPAR”, “detectably labeled recombinant hybrid suPAR” and “detectably labeled suPAR fragment” is meant a suPAR polypeptide or suPAR polypeptide/peptide fragment having an attached detectable label, e.g. as described above for detectably labeled affinity reagents. Binding is detected by standard means: e.g., by measuring the amount of label associated with (a) the solid support (if any), or (b) the precipitated analyte/binding agent complexes. In other instances, the competitive suPAR (i.e. the suPAR introduced into the test sample after incubation of the test sample with the anti-suPAR analyte affinity reagent) may be labeled with an epitope that is absent from the suPAR analyte derived from the sample of body fluid, and the detection of the binding of competitive suPAR molecule facilitated by detecting the epitope. For example, the competitive suPAR molecule may be a recombinant fusion protein which retains the ability to bind competitively to the affinity reagent used in the assay. Binding of suPAR fusion protein to the anti-suPAR affinity reagent may then be detected by incubating the sample with a detectably labeled second affinity reagent which specifically binds the fusion protein and does not bind the suPAR analyte from the sample. An example of a recombinant suPAR fusion protein is one that contains an N-terminal extension of amino acids, which recombinant suPAR fusion protein may be used in such a detection method, since affinity reagents which specifically bind to the N-terminal amino acid extension of the recombinant molecule would not be expected to bind to a suPAR analyte present in a sample. Examples of other epitopes which may be introduced into a suPAR fusion protein include epitopes for use as targets for chemical modification and epitopes which have an altered amino acid sequence relative to a naturally-occurring suPAR analyte (to provide a peptide epitope absent in a suPAR analyte). A lower level of binding of the detectably labeled suPAR in the test sample than in the negative control, e.g. a sample comprising a level of suPAR analyte comparable to that found in a healthy individual, indicates the presence of an elevated level of suPAR analyte in the test sample.

In some instances, more than one suPAR analyte-specific affinity reagents may be employed. For example, as mentioned above, in many instances, the suPAR analyte-specific affinity reagent will have specificity for an epitope found in both suPAR and uPAR, since suPAR is derived from uPAR. In other words, the suPAR analyte-specific affinity reagent will actually be specific for uPAR as well as suPAR. In some instances, it may be desirable to distinguish between suPAR and uPAR by, for example, using a first suPAR analyte-specific affinity reagent that is specific for suPAR (i.e. that detects both suPAR and uPAR) and a second affinity reagent that is specific only for uPAR (e.g. that detects the GPI anchor of uPAR). As another example, it may be desirable to employ suPAR analyte-specific affinity reagents that are capable of distinguishing between the various suPAR analyte variants, e.g. suPAR(I-III), suPAR(II-III), and uPAR(I). For example, in some embodiments a single type of affinity reagent that recognizes all variants of suPAR may be employed. However, in other embodiments it may be desirable to use different affinity reagents that recognize specific variants of suPAR. As such, in some embodiments, the subject assay of the present invention will detect the level of all variants of suPAR in a sample. In other embodiments, the subject assay of the present invention will detect the level of only one variant of suPAR in a sample.

Alternatively, non-ELISA based-methods for measuring the levels suPAR analyte in a sample may be employed. Representative examples include but are not limited to mass spectrometry, proteomic arrays, xMAP™ microsphere technology, flow cytometry, western blotting, and immunohistochemistry.

In some embodiments, the diagnosing of a diabetic kidney disease, e.g. determining whether a human subject with diabetes has a kidney disease or will develop a kidney disease, determining the severity of the kidney disease, predicting the progression of a kidney disease, monitoring the progression of the disease or the responsiveness of the individual to a course of treatment, etc. will include a step of comparing the detected level of suPAR analyte to a diabetic kidney disease reference, e.g. a reference value, a reference table, a reference sample, where the diagnosis is based on this comparison step.

For example, the detected level of suPAR analyte may be compared to a table or other source of predetermined reference values which provides information about the disease in a subject, e.g. that positively or negatively correlate to the presence of the diabetic kidney disease involving abnormal levels of suPAR analyte, a particular stage of the diabetic kidney disease involving abnormal levels of suPAR analyte, and the like, the likelihood of progression of the diabetic kidney disease based on abnormal levels of suPAR analyte, etc. For example, the table may comprise representative values for suPAR analyte as found in patients having diabetes but no kidney disease, or diabetes with diabetic kidney disease (e.g., early stage diabetic kidney disease or progressive diabeted kidney disease), or a particular type of diabetic kidney disease, etc. The values may be presented in numerical form (e.g. ranges of values), in picture form (e.g. as spots on a solid assay substrate), and the like. By comparing the observed values with these reference values, e.g. by comparing a pattern of suPAR analyte in the sample to a reference pattern or picture, characterization of the disease, e.g. confirmation of diagnosis, determination of disease state, etc., is readily made.

As another example, the detected level of suPAR analyte may be compared, e.g. experimentally compared, to the level of suPAR analyte detected in a negative and/or positive control sample (referred to herein as a reference sample) which provides information about the disease in a subject, e.g. that positively or negatively correlate to the presence of the diabetic kidney disease involving abnormal levels of suPAR analyte, a particular stage of the diabetic kidney disease involving abnormal levels of suPAR analyte, and the like, the likelihood of progression of the diabetic kidney disease based on abnormal levels of suPAR analyte, etc. By a “reference sample” it is meant an experimental sample that is representative of suPAR analyte levels that would be found in an individual that does (a positive reference sample) or does not (a negative reference sample) have diabetic kidney disease, and that may be assayed alongside the test sample as a positive or negative control. In some embodiments, the reference sample is an experimentally prepared sample (that is, a sample not acquired from an individual) that comprises a known concentration of suPAR analyte comparable to that found in a non-affected individual or an affected individual, e.g., an individual with diabetes that has not yet developed diabetic kidney disease, an individual with early stage diabetic kidney disease; an individual with progressive diabetic kidney disease, and the like. In some embodiments, the reference sample is a sample prepared from an individual, e.g. a non-affected individual or an affected individual, e.g., an individual with diabetes that has not yet developed diabetic kidney disease, an individual with early stage diabetic kidney disease; an individual with progressive diabetic kidney disease, and the like. Appropriate reference samples for the assay include samples of blood, serum, or urine collected from human subjects who do not have a diabetic kidney disease (i.e., a negative control), or samples which contain a known, predetermined amount of a suPAR analyte (i.e., a positive control). In some embodiments the level of affinity reagent binding in the test sample (that is, the sample from the individual for whom a diagnosis is being made) is compared to a range of negative and positive control samples, in which the positive control samples have a range of predetermined quantities of suPAR present, and the negative control samples do not have any suPAR present or have suPAR levels that are comparable to a non-affected control.

In some embodiments, the detected amount of suPAR analyte is used in combination with other assessments of the diabetic individual to provide a diabetic kidney disease diagnosis. For example, the amount of protein, e.g. albumin and/or creatinine, in the urine may be measured. As another example, the filtration rate of the glomeruli (glomerular filtration rate, or GFR) may be calculated. Clinical symptoms such as the frequency of urination, the retention of fluids in tissues, and blood pressure may also be assessed.

In some embodiments, providing a diagnosis of a diabetic kidney disease, e.g. a determination of whether a human subject with diabetes has a kidney disease or will develop a kidney disease, a determination of the severity of the kidney disease, a prediction of the progression of a kidney disease, a monitoring of the progression of the disease or the responsiveness of the individual to a course of treatment, etc. will include a step of generating a written report that includes the artisan's monitoring assessment, i.e. the artisan's prediction of the onset of early stage diabetic kidney disease or progressive diabetic kidney disease (a “DKD prognosis”), the artisan's diagnosis of the subject's diabetic kidney disease (a “DKD diagnosis”), the artisan's characterization of the subject's diabetic kidney disease, e.g. as early stage or progressive (a “DKD characterization”), the artisan's assessment of the progression of the diabetic kidney disease (a “DKD status report”), etc. Thus, a subject method may further include a step of generating or outputting a report providing the results of a monitoring assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a subject monitoring assessment and its results. A subject report includes at least a diabetic kidney disease prediction, diabetic kidney disease diagnosis, diabetic kidney disease characterization, or diabetic kidney disease status report, i.e. a prediction as to the likelihood of a patient developing DKD, a diagnosis of DKD, a characterization of the DKD, or a status update of DKD, respectively. A subject report can be completely or partially electronically generated. A subject report can further include one or more of: 1) information regarding the testing facility; 2) service provider information; 3) patient data; 4) sample data; 5) an assessment report, which can include various information including: a) reference values employed, b) test data, where test data can include, e.g., a suPAR analyte level determination; and c) other clinical assessments such as levels of albumin or creatinine in the urine, GFR, etc.; and 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, etc. from a subject. Data generation can include measuring the level of suPAR analyte concentration in the test individual as well as in a reference sample. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, prior episodes of symptoms of diabetic kidney disease, and any other characteristics of the diabetes), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report may include a sample data section, which may provide information about the biological sample analyzed in the monitoring assessment, such as the source of biological sample obtained from the patient (e.g. blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prediction of the likelihood that the subject will develop diabetic kidney disease. The interpretive report can include a diagnosis of diabetic kidney disease. The interpretive report can include a characterization of diabetic kidney disease. The interpretive report can include, for example, the results of a suPAR analyte detection assay (e.g., “3000 pg/ml suPAR in serum”); and interpretation, i.e. prediction, diagnosis, or characterization. The assessment portion of the report can optionally also include a recommendation(s). For example, where the results indicate that diabetic kidney disease is likely, the recommendation can include a recommendation that diet be altered, blood pressure medicines administered, etc., as recommended in the art or as described in greater detail below.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g. prediction, diagnosis or characterization of diabetic kidney disease).

### Utility

By practicing the subject methods as described herein, a diagnosis of a diabetic kidney disease condition may be made, including, for example, a determination of whether a human subject with diabetes has a kidney disease; a determination of whether a human subject with diabetes will develop a kidney disease (i.e. prognosing a diabetic kidney disease); a characterization of the diabetic kidney disease, e.g. determining the severity of the diabetic kidney disease; a prediction of the progression of the diabetic kidney disease; monitoring the progression of the diabetic kidney disease; or monitoring the responsiveness to a course of treatment therefore, etc.

As demonstrated in the Examples below, the subject methods described herein are more accurate than current methods for diagnosing, e.g. identifying, characterizing, prognosing, or monitoring, a diabetic kidney disease. For example, current methods include measuring levels of albumin in urine. However, as shown in the Examples below, diabetic individuals with normoalbuminuria, i.e. no or low levels of albumin in urine, may have diabetic kidney disease or may be at high risk for developing diabetic kidney disease. The subject methods described herein may be used to identify such individuals early and begin treatment before the condition of their kidneys worsens.

The method of the subject invention facilitates diagnosis of diabetic kidney disease prior to or coincident with the onset of clinical symptoms (e.g., decrease in GFR, microalbuminuria, macroalbuminuria, etc.). For example, the method of the subject invention may provide a diagnosis of diabetic kidney disease prior to (e.g., 1 month or more, e.g. 3 months or more, 6 months or more, 1 year or more, 2 years or more, 3 years or more, 4 years or more, or up to about 4 years prior to) onset of clinical symptoms. The diagnostic method of the subject invention is particularly advantageous over other diagnostic methods for detecting diabetic kidney disease since elevated levels of suPAR analyte precede other clinical indications, and suPAR analyte may be detected with such sensitivity. For example, in one representative embodiment, the subject methods are employed with diabetic individuals who present with normoalbuminuria and a normal or elevated GFR. Prior to the present invention, a prognosis that such an individual was at risk for developing a diabetic kidney disease would not have been possible.

In another representative embodiment, the subject methods are employed to assess or determine the severity and extent of diabetic kidney disease in a diabetic patient. Many patients with diabetic kidney disease have a progressive form of the disease that leads to organ failure. The subject methods may be employed to determine if the patient has progressive diabetic kidney disease, which may be useful in informing the caregiver as to whether to consider more aggressive treatments such as dialysis or transplantation.

Where the subject methods are employed to confirm an initial diagnosis of a diabetic kidney disease (i.e., early stage or progressive diabetic kidney disease), a sample is obtained from the individual suspected of having the disease. In such embodiments, the subject may be identified as presenting the classical symptoms of diabetic kidney disease, e.g. blood in urine, albuminuria and/or proteinuria, a decreased GFR rate, etc. or have a medical history that indicates susceptibility to diabetic kidney disease. For example, the sample is assayed for the level of suPAR analyte present, and then compared to reference, where the reference correlate a detected level amount with a diabetic kidney disease.

The subject methods are also employed to determine the severity of the diabetic kidney disease in an individual already known to have a diabetic kidney disease. In other words, the subject method can be used to determine whether the human subject suffering from a diabetic kidney disease has early stage diabetic kidney disease or progressive diabetic kidney disease. To determine the stage of the disease, the observed level of suPAR analyte in the assayed sample is compared to a reference, e.g. one or more reference values, or one or more reference samples, or that are correlated to a particular stage of the diabetic kidney disease.

In yet other embodiments, characterization of disease activity yields information concerning progression of the diabetic kidney disease in the human subject, e.g. whether progression of the disease has accelerated or slowed. For example, the initial characterization date, i.e. the level of suPAR analyte present in the sample derived from the individual, could be employed as a baseline value to evaluate subsequent testings, e.g. at some time following the initial testing, e.g. 3 months or more, 6 months or more, 1 year or more, 2 years or more, 3 years or more, 4, years or more, or 5 years or more after initial testing. If the level of suPAR analyte remains relatively constant or decreases in subsequent testing, this indicates that the diabetic kidney disease is not progressing and may be resolving. Alternatively, if the level of suPAR analyte increases, this indicates that the diabetic disease is progressing in severity.

In some embodiments, the subject methods of the present invention may be used in the treatment of an individual for a diabetic kidney disease. In such embodiments, the subject methods are employed to first determine whether a human subject suffers from a diabetic kidney disease (or the severity of the disease) by determining the level of suPAR analyte in a sample derived from the individual according the subject methods. Once a determination has been made with respect to whether the individual suffers from a diabetic kidney disease, a treatment protocol is identified for the human subject based on the determination of the level of suPAR analyte in a sample derived from the human subject.

For example, in instances in which it is determined that the individual has a diabetic kidney disease, the treatment may include administering a known therapeutic for kidney disease. One non-limiting example of such a therapeutic would be a suPAR antagonist. By a “suPAR antagonist” it is meant an agent that inhibits the activity of suPAR, e.g. by inhibiting the expression of suPAR (e.g. an siRNA or antisense RNA) or by inhibiting the binding of suPAR to podocytes and activation of podocytes by suPAR (e.g. a suPAR-specific antibody, a suPAR-specific antagonist peptide, a small molecule). See, for example, PCT Publication No. WO 2010/054189, which discloses suPAR-specific antagonistic antibodies, and Ploug et al. (Peptide-derived antagonists of the urokinase receptor. Affinity maturation by combinatorial chemistry, identification of functional epitopes, and inhibitory effect on cancer cell intravasation. Biochemistry 2001; 40: 12157-12168), which discloses peptide antagonists of uPAR/suPAR, the full disclosure of which are incorporated herein by reference.

Another non-limiting example of a therapeutic that might be prescribed would be a drug used to lower blood pressure. Such drugs have been shown to slow the progression of kidney disease significantly. Two types of drugs, angiotensin-converting enzyme (ACE) inhibitors and angiotensin receptor blockers (ARBs), have proven effective in slowing the progression of kidney disease. An example of an effective ACE inhibitor is lisinopril (Prinivil, Zestril), which has been shown to lower proteinuria and slow deterioration even in people with diabetes who did not have high blood pressure. An example of an effective ARB is losartan (Cozaar), which has also been shown to protect kidney function and lower the risk of cardiovascular events. Other therapeutic agents that control blood pressure that may be administered, include, for example, diuretics, beta blockers, calcium channel blockers, etc. Any convenient medicine that helps patients achieve a blood pressure target of 130/80 or lower provides benefits. In some instances, two or more drugs that control blood pressure may be administered.

Other treatments may also be recommended if a diabetic kidney disease is diagnosed, including, for example, the selective removal of suPAR analyte from the blood of the individual. The selective removal of constituents from the blood, e.g. cells, proteins, etc. by techniques such as apheresis is well known in the art. The removal of suPAR analyte from the blood of an individual by apheresis through a column comprising a suPAR antibody is described in greater detail in PCT Application No US2012/000240, the full disclosure of which is incorporated herein by reference.

In some instances, the treatment will include a moderate-protein or low-protein diet. In some instances, the treatment may comprise a regimen of intensive management of blood glucose or glycemic control. Intensive management of blood glucose is a treatment regimen that aims to keep blood glucose levels close to normal. The regimen includes testing blood glucose frequently, administering insulin throughout the day on the basis of food intake and physical activity, following a diet and activity plan, and consulting a health care team regularly. Some people use an insulin pump to supply insulin throughout the day. A number of studies have pointed to the beneficial effects of intensive management of blood glucose. In the Diabetes Control and Complications Trial supported by the National Institute of Diabetes and Digestive and Kidney Diseases (NIDDK), researchers found a 50% decrease in both development and progression of early diabetic kidney disease in participants who followed an intensive regimen for controlling blood glucose levels. The intensively managed patients had average blood glucose levels of 150 milligrams per deciliter-about 80 milligrams per deciliter lower than the levels observed in the conventionally managed patients. The United Kingdom Prospective Diabetes Study, conducted from 1976 to 1997, showed conclusively that, in people with improved blood glucose control, the risk of early kidney disease was reduced by a third. Additional studies conducted over the past decades have clearly established that any program resulting in sustained lowering of blood glucose levels will be beneficial to patients in the early stages of CKD. In some instances, the treatment may include dialysis or a kidney transplant.

In other embodiments, characterization data of the level of suPAR analyte present in a sample derived from an individual obtained by the subject methods may also be used to determine whether a particular therapeutic regimen is having positive affects with respect to the progression of the diabetic kidney disease. For example, at various time periods during the course of treatment, the subject methods may be performed to obtain a reading of the amount of suPAR analyte present in a sample derived form a human subject under a particular treatment regimen. If the level of suPAR analyte is increasing, this indicates that the treatment regimen is not having the desired effect, where the desired effect is to slow the progression of the diabetic kidney disease. Alternatively, if the level of suPAR analyte is decreasing, this indicates that the treatment regimen is working with respect to slowing the progression of the diabetic disease.

### Kits

Also provided are kits that find use in practicing the subject methods, as described above. The kits for practicing the subject methods at least include reagents for assaying a sample derived from a human subject for suPAR analyte, where such kits may include: suPAR analyte affinity reagents, such as an antibody, or fragment or mimetic thereof, and/or immunoassay devices comprising the same members of a signal producing system, such as antibodies, enzyme substrates, and the like; various buffers for use in carrying out the subject detection assays; and the like.

The kits may include means for obtaining the patient sample, e.g. a syringe, a urine collection cup, etc. The kits may include one or more reagents necessary for preparation of the patient derived sample, such as heparin, Ficoll-Hypaque, lysing buffer, protease inhibitor, and the like, e.g. where the patient sample is blood derived, etc. The subject kits may further include one or more components employed in fractionation of the sample, such as an electrophoretic medium or precursors thereof, e.g. dried precursors of polyacrylamide gels, one or more buffer mediums or components thereof, and the like.

In certain embodiments, the kits further include at least an information storage and presentation medium that contains reference data with which assay results may be compared in order to diagnose and/or characterize the diabetic kidney disease in the individual being assayed, i.e., reference data that that positively or negatively correlate to the presence of the diabetic kidney disease, a particular stage of the disease, a particular risk for developing the disease, and the like. The information storage and presentation medium may be in any convenient form, such as a printed information on a package insert, an electronic file present on an electronic storage medium, e.g. a magnetic disk, CD-ROM, and the like. In yet other embodiments, the kits may include alternative means for obtaining reference data, e.g. a website for obtaining the reference data “on-line.” Additionally or alternatively, the kits in certain embodiments further include one or more reference samples that are representative of suPAR analyte levels that would be found in a non-affected individual (i.e. a negative control) or an individual having a diabetic kidney disease (a positive reference sample), which could be assayed concurrently with the test sample as a control.

In certain embodiments, the kits may include one or more reagents from an additional biochemical assay which is used to detect the presence of and/or characterize the diabetic kidney disease, e.g. reagents for the detection of albumin, creatinine, GFR, etc, as known in the art.

The subject kits further typically include instructions for carrying out the subject methods, where these instructions may be present on a package insert and/or the packaging of the kit.

The kit components may be present in separate containers, or one or more of the components may be present in the same container, where the containers may be storage containers and/or containers that are employed during the assay for which the kit is designed.

### Systems

Also provided are systems that find use in practicing the subject methods, as described above. The systems for practicing the subject methods at least include reagents for assaying a sample derived from an individual for suPAR analyte, where such systems may include: suPAR analyte affinity reagents, such as an antibody, or fragment or mimetic thereof, and/or immunoassay devices comprising the same members of a signal producing system, such as antibodies, enzyme substrates, and the like; various buffers for use in carrying out the subject detection assays; a reference for determining the amount of suPAR analyte in a sample; and the like.

Furthermore, additional items that are required or desired in the protocol to be practiced with the system components may be present, which additional items include, but are not limited to: means for obtaining the patient sample, e.g. a syringe; one or more reagents necessary for preparation of the patient derived sample, such as heparin, Ficoll-Hypaque, lysing buffer, protease inhibitor, and the like; instructions for carrying out the subject methods; one or more reagents from an additional biochemical assay which is used to detect the presence of and/or characterize the diabetic kidney disease involving abnormal levels a suPAR analyte.

### Devices

Also provided are devices that find use in practicing the subject methods, as described above. Devices for practicing the subject methods at least include reagents for assaying a sample derived from a human subject for a suPAR analyte, where such devices may include: suPAR analyte affinity reagents, such as an antibody, or fragment or mimetic thereof, immobilized on the surface of a solid support.

Additional items that are required or desired in the methods to be practiced with the devices may be present, which additional items include, but are not limited to: means for obtaining the patient sample, e.g. a syringe or pricking element, one or more reagents necessary for preparation of the patient derived sample, such as heparin, Ficoll-Hypaque, lysing buffer, protease inhibitor, and the like; instructions for carrying out the subject methods using the subject devices; one or more reagents from an additional biochemical assay which is used to detect the presence of and/or characterize the diabetic kidney disease.

A number of such devices are known in the art. In one non-limiting example, the apparatus will generally employ a continuous flow-path of a suitable filter or membrane, having at least three regions, a fluid transport region, a sample region, and a measuring region. The sample region is prevented from fluid transfer contact with the other portions of the flow path prior to receiving the sample. After the sample region receives the sample, it is brought into fluid transfer relationship with the other regions, and the fluid transfer region contacted with fluid to permit a reagent solution to pass through the sample region and into the measuring region. The measuring region may have bound to it the first affinity reagent, and second labeled affinity reagent combined with the assayed sample and the sandwich assay performed as above.

In another non-limiting example the device is a dipstick, to the surface of which is bound an affinity reagent, such an antibody, or fragment or mimetic thereof, which specifically binds suPAR analyte. In one such an exemplary device, the dipstick is inserted directly into a test sample (e.g., blood, serum, or urine) derived from a human subject for a period of time sufficient to permit binding of suPAR analyte to the affinity reagent bound to the dipstick. The dipstick may be then withdrawn and, if necessary, washed to remove nonspecifically bound material. The dipstick is then inserted into a container containing a detectably labeled second affinity reagent, such an antibody, or fragment or mimetic thereof, which specifically binds suPAR analyte. After incubation for a time sufficient for binding of the second antibody to the suPAR analyte-affinity reagent complexes, the dipstick may be washed and binding of the second affinity reagent detected by standard means. Where necessary for detection of the second antibody, the dipstick may be inserted into a second container containing a reagent which activates the detectable label on the second antibody.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

A mechanism of podocytopathy is identified here. When normal podocytes in culture are exposed to the sera of patients with type 1 diabetes and diabetic kidney disease (DKD), they have impaired insulin signaling, actin remodeling, and increased apoptosis relative to podocytes cultured with sera of age and sex matched healthy controls. In contrast, podocytes cultured with sera from patients with type 1 diabetes and no DKD do not demonstrate any significant modification of insulin signaling, apoptosis or actin remodeling. Fasting blood glucose, HbA1c, duration of diabetes, total cholesterol, LDL cholesterol, and HDL cholesterol are not significantly different between the groups. These data indicate that circulating factors other than glucose and cholesterol in the serum of patients with DKD induce podocyte malfunction and death.

Soluble circulating urokinase receptor (suPAR) has recently been identified as an important mediator of proteinuria in another glomerular disorder. As demonstrated herein, suPAR levels are elevated in DKD as compared to controls, indicating that suPAR may be used as a diagnostic marker for DKD. suPAR levels are also elevated in the sera from patients with progressive DKD (n=5) who, at the time of collection, are still normoalbuminuric or microalbuminuric with normal kidney function, indicating that high circulating levels of suPAR may be used as a prognostic marker for the development of microalbuminuria in diabetic kidney disease.

Elevated Circulating suPAR Concentrations are Associated with DKD.

The sera from three groups of patient (N=10 each) were studied to determine if suPAR concentrations in blood are associated with diabetic kidney disease: patients with normal human sera (NHS); patients with Type 1 diabetes and no microalbuminuria (DM w/o DKD); and patients with type 1 diabetes and diabetic kidney disease (DM w DKD). Patients were matched for age, sex, duration of diabetes, HgA1c, and lipid profile Dot plot analysis of circulating suPAR concentrations demonstrates a significantly higher serum circulating suPAR level in patients with DKD (group 3, 3851±746 pg/ml) when compared to DM w/o DKD (group 2, 2300±576.4 pg/ml) and NHS (group 1, 1689±219.6 pg/ml, p<0.001). These data demonstrate that suPAR should be further studied as a biomarker for the diagnosis as well as for the prediction of DKD. Circulating suPAR concentration in subjects with glomerular disease and healthy human subjects. MN, membranous nephropathy. *P<0.05 for FSGS versus MN and preeclampsia; #P<0.001 for FSGS versus healthy, MCD relapse, and MCD remission.

Elevated Circulating suPAR are Observed in Early DKD Prior to any Change in GFR.

Historical sera from the year 2000 and 2007 was obtained from five patients with diabetes that have now progressed to diabetic nephropathy with altered renal function. In year 2000, two of the five patients were normolabuminuric (light gray bar), while the other three had microalbuminuria with normal renal function (dark gray). In year 2007, the two patients that were initially normoalbuminuric progressed to microalbuminuria, while those three patients that had microalbuminuria progressed to macroalbuminuria. The concentration of suPAR was higher in the sera of patients that progressed to DKD when compared to age and sex matched normal human sera (NHS) irrespectively of the time that the samples were collected, suggesting that increased circulating suPAR prior to the development of microalbuminuria may predict patients risk to progress to DKD.

In addition, five patients with T1D that ultimately developed progressive DKD with loss of GFR were studied. Two historical sets of sera were available for these 5 patients: one (Normo/Micro) collected when all patients had normal GFR and either microalbuminuria (n=3) or normoalbuminuria (n=2); a second one (Macro) collected 7 years later when all patients had developed macroalbuminuria and had still normal GFR. Ten normal human sera controls (NHS) matched for age and sex to the Normo/Micro group were utilized. Circulating suPAR concentrations were higher in patients with DKD already at the time they were normoalbuminuric and with normal GFR, therefore prior to the diagnosis of DKD. These data demonstrate that increased circulating suPAR in the population of patients with diabetes may represent an early biomarker of disease development and progression.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for determining if a diabetic individual has a diabetic kidney disease in, comprising:
detecting the amount of suPAR analyte in a blood sample from a diabetic individual, and
determining based on the amount of suPAR analyte detected if the diabetic individual has a kidney disease.

2. The method according to claim 1, wherein an amount of suPAR analyte of 3000 pg/ml or more in a diabetic individual is diagnostic of diabetic kidney disease.

3. The method according to claim 1, wherein an amount of suPAR analyte of 4000 pg/ml or more in a diabetic individual is diagnostic of progressive diabetic kidney disease.

4. The method according to claim 1, wherein the method further comprises comparing the amount of suPAR analyte detected to a reference, and determining if the diabetic individual has a kidney disease based on the comparison.

5. The method according to claim 4, wherein:
the reference is a reference sample representative of suPAR analyte levels in an individual that does not have kidney disease, wherein an elevated amount of suPAR relative to the reference is diagnostic of a diabetic kidney disease.

6. A method for providing a diabetes prognosis, comprising:
detecting the amount of suPAR analyte in a blood sample from an individual with diabetes, and
determining a prognosis for the diabetes based on the amount of suPAR analyte detected.

7. The method according to claim 6, wherein the individual has normal glomerular filtration rate (GFR) and normoalbuminuria, wherein an amount of suPAR analyte of 3000 pg/ml or more is prognostic of the development of diabetic kidney disease.

8. The method according to claim 6, wherein the individual has microalbuminuria, wherein an amount of suPAR analyte of 4000 pg/ml or more is prognostic of the development of progressive diabetic kidney disease.

9. A kit for diagnosing or prognosing diabetic kidney disease, comprising:
a suPAR analyte detection reagent, and
a diabetic kidney disease reference.

10. The kit according to claim 9, wherein the suPAR analyte detection reagent comprises a suPAR analyte affinity reagent conjugated to a solid support.

11. A method for treating diabetic kidney disease in an individual, comprising:
diagnosing diabetic kidney disease by a method according to claim 1, and
treating the individual for diabetic kidney disease.

12. The method according to claim 11, wherein the treating comprises administering an effective amount of a therapeutic agent for treating diabetic kidney disease.

13. The method according to claim 12, wherein the agent inhibits suPAR activity.

14. The method according to claim 13, wherein the agent is a suPAR-specific antibody.

15. The method according to claim 11, wherein the treating comprises apheresing the individual's blood to remove suPAR.

16. A kit for treating diabetic kidney disease, comprising:
a suPAR detection reagent, and
a therapeutic agent for treating diabetic kidney disease.

17. The kit according to claim 16, wherein the therapeutic agent is a suPAR-specific antibody.

18. A kit for treating diabetic kidney disease, comprising:
a suPAR analyte detection reagent, and
an apheresis cartridge.

19. The kit according to claim 18, wherein the apheresis cartridge comprises a suPAR analyte-specific affinity reagent.

20. The kit according to claim 18, wherein the suPAR analyte-specific affinity reagent is a suPAR-specific antibody.
